(19) United States
(12) Patent Application Publication
(10) Pub. No.: US 2005/0137141 A1
(43) Pub. Date: Jun. 23, 2005

(54) Prodrug composition

(76) Inventor: John Hilfinger, Ann Arbor, MI (US)

Correspondence Address: Avery N. Goldstein;Gifford, Krass, Groh, Sprinkle,, Anderson & Citkowski, P.C., 280 N. Old Woodward Ave., Suite 400, Birmingham, MI, 48009-5394, US

(21) Appl. No.: 10/972,729

(22) Filed: Oct. 25, 2004

Related U.S. Application Data
(60) Provisional application No. 60/514,121, filed on Oct. 24, 2003.

(51) Int. Cl. A61K 38/04; A61K 31/7076; A61K 31/7072; A61K 31/573; A61K 31/4745; A61K 31/485

(52) U.S. Cl. 514/19; 514/46; 514/49; 514/263.3; 514/492; 514/282; 514/34; 514/262.1; 514/317; 514/171; 514/167; 514/389; 514/617; 514/640; 514/561

(57) ABSTRACT

A prodrug composition is provided which includes a pharmaceutical species and an amino acid having a covalent bond to the pharmaceutical species. The pharmaceutical species is characterized by bioavailability of 30% or less and a molecular weight in the range of 100-1000 Daltons. The composition is characterized further in that the pharmaceutical species is not acyclovir, ganciclovir, BRL44385, or penciclovir. Also described is an inventive method of delivering a pharmaceutical species to an individual including the step of orally administering an inventive prodrug to an individual. In one embodiment the prodrug includes a pharmaceutical species characterized by bioavailability of 30% or less, wherein the pharmaceutical species has a molecular weight in the range of 100-1000 Daltons. The inventive prodrug is transported from the gastrointestinal lumen by a specific transporter and is enzymatically cleaved to yield the pharmaceutical species, such that the pharmaceutical species is delivered to the individual.

### REFERENCE TO RELATED APPLICATIONS

This application claims priority of U.S. Provisional Patent Application 60/514,121 filed Oct. 24, 2003, the entire content of which is incorporated herein by reference.

### FIELD OF THE INVENTION

The present invention generally relates to prodrugs that are substrates for enzymatic cleavage, and in particular to prodrugs where the enzymatic substrate portion of the prodrug is simultaneously a substrate for a membrane transporter.

### BACKGROUND OF THE INVENTION

A prodrug in vivo activation strategy is considered attractive in increasing the concentration of an active compound at the local site of enzymatic cleavage to an active compound with the concurrent limitation of systemic exposure to the active compound so as to reduce side effects. It is conventional to couple a moiety to an active drug species such that an enzyme associated with the target site acts on the substrate moiety to generate an active species at a desired locality. Enzymes useful in prodrug activation have been described and include enzymes such as thymidine kinase, cytosine deaminase, and purine nucleoside phosphorylase, as described in U.S. Pat. Nos. 5,338,678; 5,552,311; 6,017,896; and 6,027,150. While the basic concept of coupling a substrate moiety to an active species is well known, this approach has met with limited success owing to difficulty in transporting the prodrug into a particular type of cell, and the presence of a cleavage enzyme in cell types other than those targeted for therapeutic interaction with the active drug species. Thus, there exists a need for a prodrug where the enzymatic cleavage substrate bound to the active drug species also serves as a membrane transporter species.

### SUMMARY OF THE INVENTION

A prodrug composition is provided which includes a pharmaceutical species and an amino acid having a covalent bond to the pharmaceutical species. The pharmaceutical species is characterized by bioavailability of 30% or less and a molecular weight in the range of 100-1000 Daltons. The composition is characterized further in that the pharmaceutical species is not acyclovir, ganciclovir, BRL44385, or penciclovir.

In a further embodiment, a prodrug composition is provided in which includes a pharmaceutical species and an amino acid having a covalent bond to the pharmaceutical species wherein the pharmaceutical species is selected from the group consisting of: floxuridine, gemcitabine, cladribine, melphalan, and cidofovir.

Also described is an inventive method of delivering a pharmaceutical species to an individual which includes the step of orally administering an inventive prodrug to the gastrointestinal lumen of an individual. In one embodiment the prodrug includes a pharmaceutical species characterized by bioavailability of 30% or less, wherein the pharmaceutical species has a molecular weight in the range of 100-1000 Daltons. The method is further characterized in including the step of administering a compound as detailed herein wherein the pharmaceutical species included in the composition is not acyclovir, ganciclovir, BRL44385, or penciclovir. The inventive prodrug is transported from the gastrointestinal lumen by a specific transporter and is enzymatically cleaved to yield the pharmaceutical species, such that the pharmaceutical species is delivered to the individual.

### BRIEF DESCRIPTION OF THE DRAWINGS

FIG. 1 is a graph showing floxuridine prodrug analog uptake as mediated by HPEPT1 infected cells as compared to normal cells with 3′ and 5′ valyl esters of floxuridine showing enhanced uptake in HPEPT1/Hela cells with uptake measured for each of the synthesized prodrugs. Cephalexin, a known drug transported by HPEPT1, is included as a positive control; and

FIG. 2 is a graph showing activation of floxuridine amino acid prodrug.

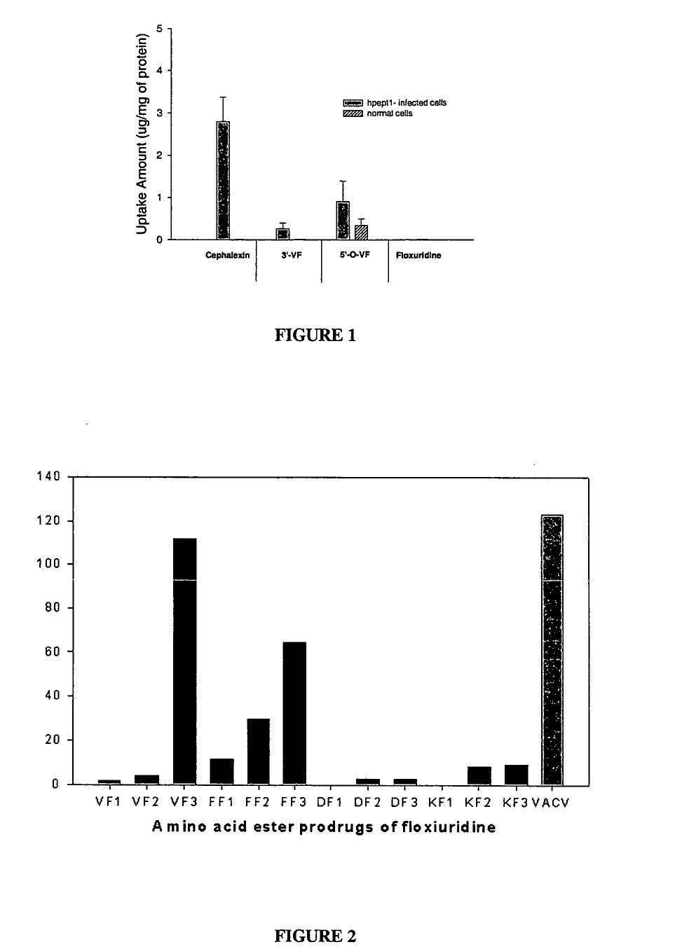

### DETAILED DESCRIPTION OF THE INVENTION

The prodrugs which are the subject of the present invention include prodrugs that contain a pharmaceutical species (X) for the treatment of a disease state and a promoiety (Y) that is covalently bound to the pharmaceutical species where the promoiety Y is an enzymatic substrate as well as a substrate for a membrane transporter. The present invention has utility as a therapeutic agent for the treatment of a variety of disease states.

An inventive prodrug enhances the bioavailability of the pharmaceutical species while specifically targeting the enzyme responsible for promoiety removal and thus pharmaceutical species release. Bioavailability is defined herein as the amount of drug systemically available in comparison to the total amount of drug delivered to an individual. Bioavailability is typically expressed as % bioavailability and is generally measured by comparing plasma levels of drug after oral administration to plasma levels of drug after intravenous administration. This definition includes first pass metabolism, that is gut and liver metabolism, which when it occurs, occurs before the drug is available systemically. Thus, highly metabolized drugs may be completely absorbed but have a bioavailability less than 100%. Bioavailability is directly related to the fraction of a drug absorbed or “fraction absorbed”, which refers to the percent of a total orally delivered drug dose transported or diffused across the luminal membranes of the gastrointestinal tract into the portal vein.

A prodrug according to the present invention has the general form X-Y. X includes a wide variety of pharmaceutical compounds that have accessible reactive groups to which a promoiety is covalently bonded. In a preferred embodiment, an inventive prodrug includes a pharmaceutical species X having bioavailability of 30% or less. Covalent bonding of a promoiety to the pharmaceutical species X enhances bioavailability of the pharmaceutical species by greater than 2 fold. An exemplary list of pharmaceutical species X that currently have clinical indications and bioavailability of 30% or less is described herein.

In one embodiment, the pharmaceutical species X has a bioavailability of 30% or less and a molecular weight ranging from 100-1000 Daltons. In another embodiment of an inventive prodrug, the pharmaceutical species X has a molecular weight ranging from 260-800 Daltons. In general, bioavailability of pharmaceutical species decreases with increasing molecular weight. Thus, it is surprising that modification of a pharmaceutical species X having higher molecular weights, such as those in the range of 260-1000 Daltons, enhances bioavailability.

In another embodiment, the pharmaceutical species is a cyclic nucleoside analog having a bioavailability of 30% or less.

In a further embodiment, the pharmaceutical species X includes a halogen. Examples of pharmaceutical species (X) according to the present invention illustratively include anti-neoplastic compounds such as floxuridine, gemcitabine, cladribine, dacarbazine, melphalan, mercaptopurine, thioguanine, cis-platin, and cytarabine; and anti-viral compounds such as fludarabine, cidofovir, tenofovir, and pentostatin. Further examples of pharmaceutical species according to the invention include adenocard, adriamycin, allopurinol, alprostadil, amifostine, aminohippurate, argatroban, benztropine, bortezomib, busulfan, calcitriol, carboplatin, daunorubicin, dexamethasone, topotecan, docetaxel, dolasetron, doxorubicin, epirubicin, estradiol, famotidine, foscarnet, flumazenil, fosphenytoin, fulvestrant, hemin, ibutilide fumarate, irinotecan, levocarnitine, idamycin, sumatriptan, granisetron, metaraminol, metaraminol, methohexital, mitoxantrone, morphine, nalbuphine hydrochloride, nesacaine, oxaliplatin, palonosetron, pamidronate, pemetrexed, phytonadione, ranitidine, testosterone, tirofiban, toradol, triostat, valproate, vinorelbine tartrate, visudyne, zemplar, zemuron, and zinecard.

The promoiety Y is selected to be covalently bindable to the pharmaceutical species X, as well as simultaneously being a substrate for enzymatic cleavage and itself or as X-Y being a substrate for a membrane transporter. The promoiety Y includes synthetic and naturally occurring amino acids, di- and polypeptides, pentose sugars, hexose sugars, disaccharides, polysaccharides, C₂-C₂₀ linear or branched alkyl groups, and C₃-C₂₀ alkyl groups having a substituent where the substituent is selected from the group consisting of: amino, hydroxyl, phospho-, phosphatidyl-, and the aforementioned groups.

For the enhanced transport, there are a wide variety of known intestinal and cellular transporters that have been identified that could serve as targets for an inventive prodrug. A list of exemplary transporters is given in Table 2. Also in Table 2 is compiled a list of compounds that are known to interact with specific transporters.

TABLE 2
Transporter Targets.

| Transporters | Active species/substrates |
|---|---|
| Amino acid | gabapentin, D-cyclosporin, isobutyl gaba, L-methyldopa, |
| transporters | L-dopa, baclofen |
| Peptide transporter | β-lactam antibiotics, ACE inhibitors, valacyclovir, |
| (HPEPT1, HPT1) | valganciclovir, cyclosporin, L-methyldopa, cephalexin |
| Nucleoside | zidovudine, zalcitabine cladribine ara-C, ara-A, |
| transporters | fludarabine, dilazep, dipyridamole, draflazine hypoxanthine |
| (CNT1 CNT2, ENT1 | |
| ENT2) | |
| Organic cation | tetraethylammonium, N-methylnicotineamide, thiamine, |
| transporters | tyramine, tryptamine, choline, spermine, spermidine, |
| (OCT1, ORCTL3) | d-tubocurarine, procanamide, dobamine, noradrenaline, |
| | serotonin, istamine, corticosterone, MPP, despramine, |
| | qunidine, verapamil, midazolam |
| Organic anion | methotrexate, cefodizime, ceftriaxone, pravastatin, |
| transporters | temocaprilat, salicylic acid, p-amnobenzoic acid, benzoic |
| (MOAT (MRP2), MCT1) | acid, nicotinic acid, lactate |
| Glucose transporters | p-nitrophenyl-β-D-glucopyranoside, β-D- |
| (GLUT2, GLUT5, | galactopyranoside |
| SGLT1, SAAT1) | |
| Bile acid transporters | Thyroxine, chlorambucil, crilvastatin |
| (IBAT/ISBT) | |
| Phosphate transporters | fosfomycin, foscarnet, digoxin, cyclosporin |
| (NPT4, NAPI-3B, P-gp) | |
| Vitamin transporters | reduced vitamin C, methotrexate, nicotinic acid, thiamine, vitamin |
| (SVCT1-2, folate | B-12, R.I.-K(biotin)-Tat9 |
| transporters, SMVT) | |

The chemical synthesis of an inventive prodrug is appreciated to be largely dictated by the reactive sites available on the active species X or those incorporated therewith, and the corresponding reactive site found on the promoiety Y. By way of example, a pharmaceutical species X including or chemically modified to include a carboxylic acid group readily forms a covalent bond with a promoiety Y through conventional organic chemistry reactions. For instance, reaction of a pharmaceutical species carboxylic acid group with vinyl chloride creates an active species carbonyl chloride which upon reaction with a promoiety hydroxide or primary amine yields X-Y in the form of an ester (XCOOY) and an amide (XCONHY), respectively. In a similar fashion a pharmaceutical species containing a hydroxyl group is readily esterified through a similar reaction scheme. Additionally, an amine group found in a pharmaceutical species is readily alkylated by reaction with a promoiety halide to yield XNHY where the halide acid represents the other metathesis reaction product. Illustrative linkages between and X and Y include an ester, an amide, an ether, a secondary amine, a tertiary amine, and an oxime. While the synthesis of an inventive prodrug is detailed above with chemistry being performed on the pharmaceutical species X in order to form a covalent bond with a subsequent reactant promoiety Y, it is appreciated that modifying chemistry is readily performed on the promoiety Y followed by subsequent reaction with active species X. Additionally, it is appreciated that protecting agents are operative herewith to preclude reaction at one or more active sites within a pharmaceutical species X and/or promoiety Y during the course of a coupling reaction. Additionally, a deprotecting agent is operative herein to convert a pharmaceutical species X and/or a promoiety Y into a reactive thiol, amine or hydroxyl substituent. Protecting agents and deprotecting agents are well known in the art. Theodore W. Green and Peter G. M. Wets, Protective Groups in Organic Synthesis, 2^{nd} Edition (1991).

In a preferred embodiment, an inventive composition includes a prodrug having the general formula X-Y wherein an active species X is a pharmaceutical species characterized by lack of bioavailability when administered orally to an individual. In this embodiment, promoiety Y is an amino acid having a covalent bond to the pharmaceutical species. In a preferred embodiment, an inventive prodrug includes a pharmaceutical species having bioavailability of 30% or less. Covalent bonding of a promoiety to the pharmaceutical species enhances bioavailability of the pharmaceutical species by greater than 2 fold.

Naturally-occurring or non-naturally occurring amino acids are used to prepare the prodrugs of the invention. In particular, standard amino acids suitable as a prodrug moiety include valine, leucine, isoleucine, methionine, phenylalanine, asparagine, glutamic acid, glutamine, histidine, lysine, arginine, aspartic acid, glycine, alanine, serine, threonine, tyrosine, tryptophan, cysteine and proline. Particularly preferred are L-amino acids. Optionally an included amino acid is an alpha-, beta-, or gamma-amino acid. Also, naturally-occurring, non-standard amino acids can be utilized in the compositions and methods of the invention. For example, in addition to the standard naturally occurring amino acids commonly found in proteins, naturally occurring amino acids also illustratively include 4-hydroxyproline, γ-carboxyglutamic acid, selenocysteine, desmosine, 6-N-methyllysine, ε-N,N,N-trimethyllysine, 3-methylhistidine, O-phosphoserine, 5-hydroxylysine, ε-N-acetyllysine, ω-N-methylarginine, N-acetylserine, γ-aminobutyric acid, citrulline, ornithine, azaserine, homocysteine, β-cyanoalanine and S-adenosylmethionine. Non-naturally occurring amino acids include phenyl glycine, meta-tyrosine, para-amino phenylalanine, 3-(3-pyridyl)-L-alanine, 4-(trifluoromethyl)-D-phenylalanine, and the like.

In one embodiment of an inventive compound, the amino acid covalently coupled to the pharmaceutical species is a non-polar amino acid such as valine, phenylalanine, leucine, isoleucine, glycine, alanine and methionine.

In a further embodiment, more than one amino acid is covalently coupled to the pharmaceutical species. Preferably, a first and second amino acid are each covalently coupled to separate sites on the pharmaceutical species. Optionally, a dipeptide is covalently coupled to the pharmaceutical species.

An inventive prodrug is metabolized in the individual to yield the pharmaceutical species and an amino acid. For example, endogenous esterases cleave a described inventive prodrug to yield the pharmaceutical species and amino acid. Table 3 details a nonlimiting list of activation enzymes that are operative to activate various embodiments of prodrugs X-Y by removal of the prodrug moiety Y.

TABLE 3
Activation Enzymes for Inventive Prodrugs.
α/β hydrolase fold family

| | |
|---|---|
| Acylaminoacyl peptidase (EC 3.4.19.1) | Oligopeptidase B (EC 3.4.21.83) |
| Prolyl oligopeptidase (EC 3.4.21.26) | Biphenyl hydrolase-like enzyme |
| lecithin:cholesterol acyltransferase | epoxide hydrolase |
| dipeptidyl peptidase IV (DPP IV) | |

Other peptidases

| | |
|---|---|
| prolidase | prolyl aminopeptidase |
| metalloendopeptidases | tripeptidyl peptidase II |

Alkaline phosphatases and other esterases

| | |
|---|---|
| carboxylesterase | carboxylesterase |
| palmitoyl protein thioesterase | esterase D |
| intestinal alkaline phosphatase | |

Cytochrome p450s

| | |
|---|---|
| cytochrome P450IIA3 (CYP2A3) | cytochrome P(1)-450 |
| cytochrome P450 (CYP2A13) | cytochrome P-450 (P-450 HFLa) |
| cytochrome P450-IIB (hIIB1) | cytochrome P450 4F2 (CYP4F2) |
| cytochrome P4502C9 (CYP2C9) | vitamin D3 25-hydroxylase |
| cytochrome P4502C18 (CYP2C18) | lanosterol 14-demethylase |
| | cytochrome P450 (CYP51) |
| cytochrome P4502C19 (CYP2C19) | cytochrome P450 reductase |
| cytochrome P-450IID | cytochrome P450 PCN3 gene |
| cytochrome P450 monooxygenase | |
| CYP2J2 | |

In some embodiments of an inventive compound, cleavage of the bond between X and Y yields an inactive pharmaceutical species which is further metabolized in vivo to achieve the active pharmaceutical species. For example, 6-mercaptopurine and 6-thioguanine are each inactive and require phosphorylation by the enzyme hypoxanthine-guanine phosphoribosyltransferase for transformation to the active cytotoxic form.

It is appreciated that prodrugs according to the present invention are readily created to treat a variety of diseases illustratively including metabolic disorders, cancers, and gastrointestinal disease. In a preferred embodiment, an inventive prodrug is formulated for administration to a human individual, and bioavailability and fraction absorbed measurements refer to measurements made in humans. However, it is appreciated that an inventive prodrug and method of treatment may be indicated in non-human applications as well. Thus, an inventive prodrug is advantageously administered to a non-human organism such as a rodent, bovine, equine, avian, canine, feline or other such species wherein the organism possesses an enzyme and a membrane transporter for which the prodrug is a substrate.

A method of treatment according to the present invention includes administering a therapeutically effective amount of an inventive prodrug to an organism possessing an enzyme and a membrane transporter wherein the prodrug is a substrate for both.

In a particular embodiment of an inventive method for delivering a pharmaceutical species to an individual the method includes the step of administering an inventive prodrug as described herein to the gastrointestinal lumen of an individual. Particularly preferred is a prodrug which includes a pharmaceutical species characterized by bioavailability of 30% or less, a molecular weight in the range of 100-1000 Daltons, and wherein the pharmaceutical species is not acyclovir, ganciclovir, BRL44385, or penciclovir. An amino acid is included which has a covalent bond to the pharmaceutical species. The prodrug is transported from the gastrointestinal lumen by a specific transporter and enzymatically cleaved to yield the pharmaceutical species, thereby delivering the pharmaceutical species to the individual.

Variable dosing regimens are operative in the method of treatment. While single dose treatment is effective in producing therapeutic effects, it is noted that longer courses of treatment such as several days to weeks have previously been shown to be efficacious in prodrug therapy (Beck et al., Human Gene Therapy, 6:1525-30 (1995)). While dosimetry for a given inventive prodrug will vary, dosimetry will depend on factors illustratively including target cell mass, effective active species X cellular concentration, transporter efficiency, systemic prodrug degradation kinetics, and secondary enzymatic cleavage that reduces active species lifetime. It is appreciated that conventional systemic dosimetry is not applicable to the present invention.

A prodrug is administered by a route determined to be appropriate for a particular subject by one skilled in the art. For example, the prodrug is administered orally; parentally, such as intravenously; by intramuscular injection; by intraperitoneal injection; intratumorally; transdermally; or rectally. The exact dose of prodrug required is appreciated to vary from subject to subject, depending on the age, weight and general condition of the subject, the severity of the disease being treated, the particular pharmaceutical species, the mode of administration, and the like. An appropriate dose is readily determined by one of ordinary skill in the art using only routine experimentation given the teachings herein. Generally, dosage is in the range of about 0.5-500 mg per m².

Depending on the intended mode of administration, the prodrug can be in pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. Time release preparations are specifically contemplated as effective dosage formulations. The compositions will include an effective amount of the selected substrate in combination with a pharmaceutically acceptable carrier and, in addition, may include other medicinal agents, pharmaceutical agents, carriers, or diluents. Further, a prodrug may be formulated as a pharmaceutically acceptable salt.

For solid compositions, conventional nontoxic solid carriers include, for example, pharmaceutical grades of mannitol, lactose, starch, magnesium stearate, sodium saccharine, talc, cellulose, glucose, sucrose and magnesium carbonate. Liquid pharmaceutically administrable compositions can, for example, be prepared by dissolving or dispersing an active compound with optimal pharmaceutical adjuvants in an excipient, such as water, saline, aqueous dextrose, glycerol, or ethanol, to thereby form a solution or suspension. If desired, the pharmaceutical composition to be administered may also contain minor amounts of nontoxic auxiliary substances such as wetting or emulsifying agents, pH buffering agents, for example, sodium acetate or triethanolamine oleate. Actual methods of preparing such dosage forms are known, or will be apparent, to those skilled in this art; for example, see Remington's The Science and Practice of Pharmacy (20^{th} Edition).

For oral administration, fine powders or granules may contain diluting, dispersing, and/or surface active agents, and may be presented in water or in a syrup, in capsules or sachets in the dry state or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration is generally by injection. Injectables can be prepared in conventional forms, either liquid solutions or suspensions, solid forms suitable for solution or prior to injection, or as suspension in liquid prior to injection or as emulsions.

The example presented below is intended to illustrate a particular embodiment of the invention and is not intended to limit the scope of the specification, including the claims, in any way.

### EXAMPLES

### Example 1

Method for Synthesis of Floxuridine Prodrugs

Floxuridine is fluorinated pyrimidine compound that is currently used as an anti-neoplastic anti-metabolite. The drug is absorbed orally to a certain extent, but the absolute bioavailability shows high variability (Van Der Heyden S A, Highley M S, De Bruijn E A, Tjaden U R, Reeuwijk H J, Van Slooten H, Van Oosterom A T, Maes R A. Pharmacokinetics and bioavailability of oral 5′-deoxy-5-fluorouridine in cancer patients Br J Clin Pharmacol. 1999 April; 47(4):351-6.). To address the question of targeting of drugs to specific transporters within the intestine and targeted activation, a number of floxuridine amino acid ester prodrugs are synthesized, as shown in the figure.

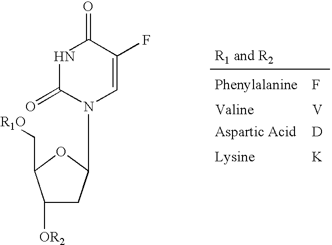

The 3′-monoester, 5′-monoester, and 3′,5′-diester prodrugs of floxuridine are synthesized as follows: N-t-Boc-amino acid (1.8 mmole), dimethyl-pyrindin-4-yl-amine (0.19 mmole) and dicyclohexyl carbodiimide (2.17 mmole) are added to floxuridine (1.33 mmole) in dry dimethylformamide (DMF) (30 ml). The solution is stirred under a nitrogen atmosphere at ambient temperature for 48 hrs and then the mixture is filtered. The DMF is removed from the filtrate in vacuo and the residue is chromatographed on silica gel, using CH₃OH/CH₂Cl₂ (1:4) as the eluant. After evaporation of the desired fractions, the resulting white solid intermediate is dissolved in 10 ml of freshly distilled trifluoroacetic acid/CH₂Cl₂ (1:1) and stirred at 0° C. for 30 min. The excess acid is removed in vacuo. The residue is freeze-dried to obtain the desired prodrug as a hygroscopic, fluffy white solid. The structures are confirmed by ¹H-NMR, ¹³C-NMR and LC/MS/MS spectrometer. For each amino acid of Equation 1, three prodrugs were synthesized: a 5′ ester, a 3′ ester and a 5′,3′ ester. The structures are confirmed by ¹H-NMR, ¹³C-NMR and LC/MS/MS spectrometer.

### Example 2

Method of Synthesis for Melphalan Prodrugs

Melphalan is a phenylalanine derivative of nitrogen mustard, a bifunctional alkylating agent active against certain human neoplastic diseases. It is absorbed orally to a certain extent, but the oral bioavailability shows high variability (Physicians Desk Reference 57^{th} edition, Thompson P D R, Montvale, N.J.). A prodrug of the melphalan containing an additional amino acid can be synthesized to increase the bioavailability of the melphan and 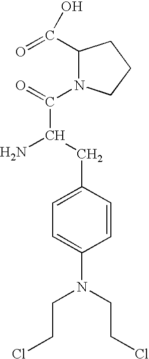
to aid in the targeting of the melphalan to the tumor tissue. An amino acid prodrug of the melphalan using proline as the amino acid is using a 4 step process.

First, t-Boc protected L-melphalan, 2 is synthesized by adding di-tert-butyl dicarbonate (196 mg, 0.89 mmol) to an ice-cold solution of melphalan (1-250 mg, 0.82 mmol) in a mixture of dioxane (2 mL), distilled water (1 mL), and 1N NaOH (1 mL). The mixture is stirred for 1 h at 0° C. and then for 16 h at room temperature. After the reaction is complete, the mixture is concentrated and ethyl acetate and distilled water are added. The pH 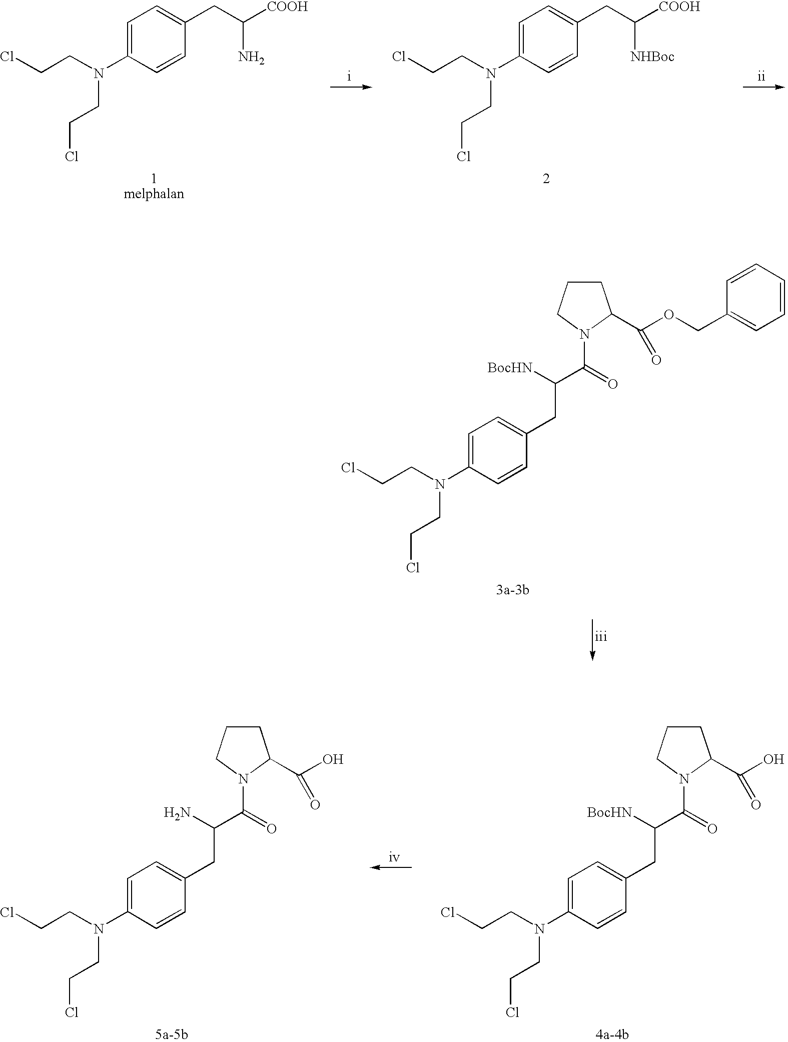
of the mixture is adjusted to 2 with hydrochloric acid and the aqueous phase is then extracted with ethyl acetate (3 times with 15 ml). The combined organic phases are washed with distilled water and brine, dried over MgSO4, and the filtrate is concentrated under vacuum to yield compound 2 (330 mg, yield 98%).

In the second step, 4-[bis(2-chloroethyl) butyloxycarbonyl]-L-phenylalanyl-L-proline benzyl ester, 3a, is synthesized by addition of compound 2 (330 mg, 0.82 mmol) to 0° C. solution of L-proline benzyl ester hydrochloride (197 mg, 0.82 mmol) dissolved in chloroform (15 mL) and triethylamine (0.14 mL). Dicyclohexylcarbodiimide (DCC, 165 mg, 0.82 mmol) is added to the mixture and it is stirred for 3 h at 0° C., allowed to warm to room temperature and stirred for an additional 24 h. The reaction mixture is filtered and the chloroform removed under reduced pressure. The residue is extracted with ethyl acetate and washed with distilled water and brine. The organic layer is dried over MgSO4 and concentrated under vacuum. The residue is subjected to column chromatography to yield compound 3a (545 mg, yield 75%). In the third step, compound 3a (520 mg, 0.88 mmol) is dissolved in 15 ml of anhydrous ethanol and 80 mg of 10% Pd/C is added. The mixture is vigorously stirred under hydrogen at room temperature for 12 h. The catalyst is removed by filtration through a bed of celite and washed with ethanol. The resulting filtrate is concentrated under vacuum and 4-[bis(2-chloroethyl)butyloxycarbonyl]-L-phenylalanyl-L-proline, 4a, is purified using a silica gel column eluted using a graded series of methylene chloride/methanol mixtures (ratios graded from 10:1 to 1:1) as the elutant. In the final step, a solution of 4a (300 mg, 0.6 mmol) in 5 ml hydrogen chloride-saturated dioxane is stirred for 25 min at 20° C. The mixture is concentrated under vacuum and the residue washed with pentane, to yield 4-[bis(2-chloroethyl)]-L-phenylalanyl-L-proline, 5a as the hydrochloride salt (210 mg, yield 80%). In this example, 4-[bis(2-chloroethyl)]-L-phenylalanyl-D-proline, 5b is synthesized by substituting D-proline benzyl ester (3b) in place of 3a and following the steps outlined above. The structures are confirmed by ¹H-NMR, ¹³C-NMR and LC/MS/MS spectrometer.

### Example 3

Synthesis of the Poorly Absorbed Nucleoside Prodrugs: Cladribine and Gemcitabine

Gemcitabine is a pyrimidine nucleoside analog and cladribine is a purine nucleoside analog. These drugs are both useful as anticancer agents. However, both drugs show very low oral bioavailability and are administered by i.v. infusion. To aid the oral pharmacokinetic and pharmacodynamic profile of the drugs such that they could be used in an oral drug product, amino acid prodrugs of these nucleoside analog drug that target the intestinal transporters can be synthesized using a two-step process. An example of the synthetic route is shown to make valyl, isoleucyl, and phenylalanyl prodrugs of Gemcitabine. Similar reaction amounts and steps can be used to synthesize the cladribine prodrugs.

In the first step, Boc protected amino acids (Boc-L-Val-OH, Boc-D-Val-OH, Boc-L-Phe-OH, Boc-D-Phe-OH, or Boc-L-Ile-OH) (1.5 mmol), dicyclohexylcarbodiimide (DCC) (1.5 mmol) and dimethylaminopyridine (DMAP) (0.15 mmol) are reacted with gemcitabine (1.5 mmol) in 10 ml of dry N,N-dimethylformamide (DMF). The reaction mixture is stirred at room temperature for 24 h. Each reaction yields three products (3′ and 5′ monoesters and 3′,5′ diesters). The reaction mixture is filtered and the DMF is removed in vacuo at 50-55° C. The residue is dissolved in ethyl acetate (30 ml) and is washed with water (2×20 ml), saturated NaHCO₃ (2×20 ml), and brine (1×20 ml). The organic layer is dried over MgSO₄ and concentrated in vacuo. The three intermediates are purified using silica gel column chromatography, which is eluted with a graded series of ethyl acetate:hexane mixtures (ethyl acetate:hexane, 1:1-1:0) as the elutant.

In the second step, the blocking group is removed from the purified intermediates by treating with 4 ml of TFA:DCM:water (6:3:1) for 4 hours. Finally, the solvent is removed under vacuum and the residue is reconstituted in water and freeze dried. The combined yield of gemcitabine prodrugs is approximately 40%. The structures are confirmed by ¹H-NMR and LC/MS/MS spectrometer.

### EXAMPLE 4

Method of Synthesis for Cidofovir Prodrugs

Cidofovir is a polar antiviral agent that exhibits very poor oral bioavailability. In order to increase the oral bioavailability of cidofovir, amino acid ester prodrugs are synthesized. In one embodiment, these prodrugs are synthesized through a modification of synthetic schemes for the synthesis of the parent drug, cidofovir. (Brodfuehrer, P. R. e.a., A Practical Synthesis of (S)—HPMPC. Tet Lett, 1994. 35(20): p. 3243-3246; and Vemishetti, P., P. R. Brodfuehrer, H. Howell, and S. C., Process for the preparation of nucleotides. 1995, Institute of Organic Chemistry and Biochemistry of the Academy of New York: USA.). As shown in scheme 1, the phosphono group remains free whereas in the second example, shown in scheme 2, the phosphono group is ethylated. In both cases amino acids are attached to the free hydroxyl group of cidofovir.

Cidofovir amino acid prodrugs with free phosphate hydroxyl groups are synthesized as described in Scheme 1. Briefly, the free amine of cytosine is protected with tert-butyloxycarbonyl (Boc) group. The Boc protected cytosine is coupled to Mtt (4-methyltrityl) protected (R)-glycidol (1), in presence of catalytic amount of sodium hydride in DMF at 105° C. for 5 h to yield compound 6. Reaction of 6 with dibenzyltosyloxymethylphosphonate (4) in presence of NaH yields the nucleotide ester (7). Removal of the Mtt group by 50% acetic acid in DCM gives the corresponding alcohol (8). The free hydroxyl group of 8 is coupled to N-tBoc-protected amino acids in presence of N,N′-dicyclohexylcarbodiimide (DCC) and dimethylamino pyridine (DMAP). The resulting Boc protected amino acid esters of cidofovir (9) is purified by column chromatography. Boc and benzyl groups are cleaved simultaneously by treating the purified material (9) with 90% trifluoroacetic acid (TFA) for 4 h. After evaporation of TFA, the residue is reconstituted with water and lyophilized. The amino acid prodrugs of cidofovir (10) are obtained as TFA salts.

Cidofovir amino acid prodrugs that also have the phosphono hydroxyls protected by ethyl groups are synthesized as described in Scheme 2. Briefly, the free amine of cytosine is protected with benzyloxy-carbonyl (Z) group. The Z protected cytosine is coupled to Mtt (4-methyltrityl) protected (R)-glycidol (1), in presence of catalytic amount of sodium hydride in DMF at 105° C. for 5 h to yield compound 11. Reaction of 11 with diethyltosyloxymethylphosphonate (5) in the presence of NaH gives the nucleotide ester (12). Removal of the Mtt group by 50% acetic acid in DCM yields the corresponding alcohol (13). The free hydroxyl group of 13 is coupled to N-Z-protected amino acids in the presence of N,N′-dicyclohexyl-carbodiimide (DCC) and dimethylamino pyridine (DMAP). The resulting Z protected amino acid esters of cidofovir (14) is purified by column chromatography. The benzyl groups are cleaved by treating the purified material (14) by hydrogenation in presences of Palladium (O). After filtration and evaporation of solvents the residue is reconstituted with water and lyophilized. The amino acid prodrugs of cidofovir (15) are obtained as HCl salts.

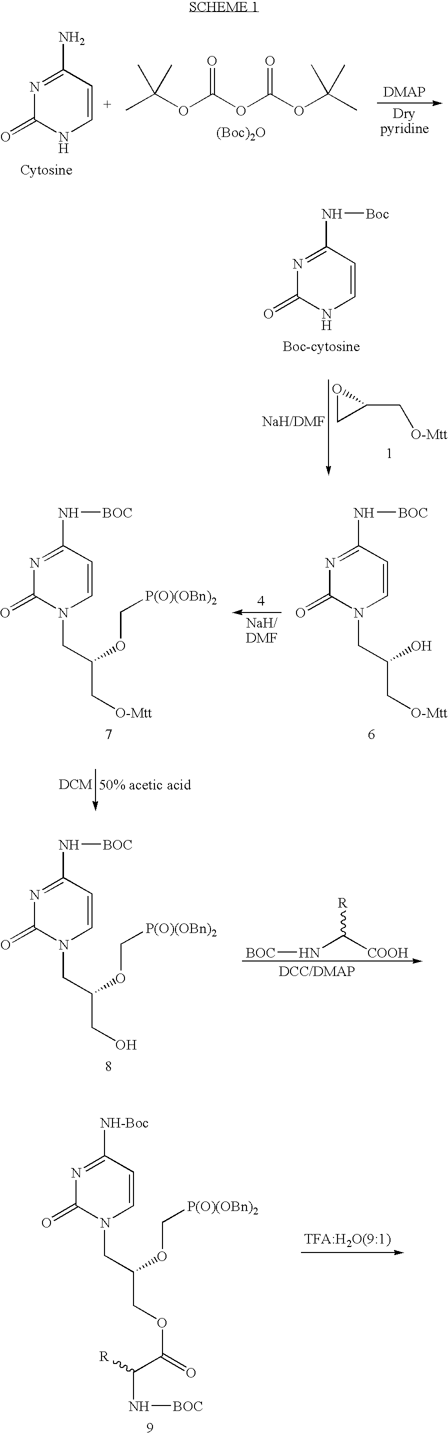

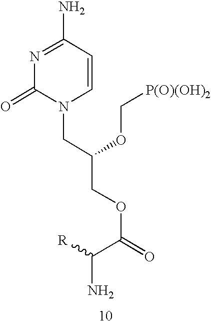

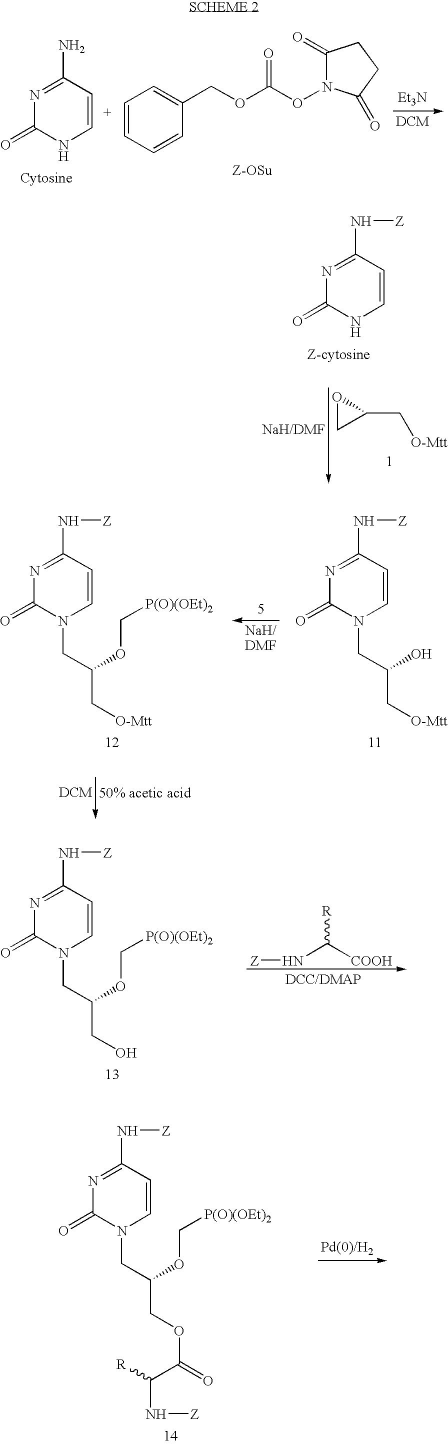

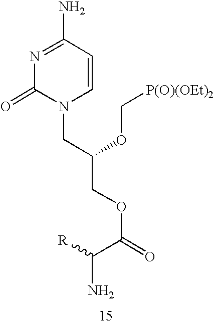

### Example 5

Determination of Binding Affinity of Amino Acid Prodrugs for the Intestinal Peptide Transporter

Amino acid prodrugs are tested for their interaction with the dipeptide transporter, HPEPT1, using tissue culture cells that are engineered to overexpress HPEPT1. In this example, the cells that overexpress HPEPT1, termed DC5, are a human meduloblastoma cell line that is stably transfected with a eukaryotic expression vector encoding HPEPT1. In this assay, the ability of the prodrug to competitively inhibit the uptake of a known substrate of HPEPT1 is measured. The known substrate is the dipeptide Glycine-Sarcosine (Gly-Sar) that has a radioactive label. DC5 cells are plated at a density of 12,000 cells/well in 96-well tissue culture plates and allowed to grow for 2 days. The cells are washed once with 200 microliters of uptake buffer and aspirated. The plates are cooled to 4° C. and 25 ul of uptake buffer containing 125 nanomoles Gly-Sar (at a specific activity of 1 microcurie/micromole) is added. The uptake buffer also contains the prodrugs to be tested at concentrations ranging from 10 micromolar to 20 millimolar. The assay is initiated by placing the plate in a shaking water bath at 37° C. and is terminated after 10 min by rapid washing with multiple changes of 4° C. phosphate buffered saline (PBS). The radioactive Gly-Sar peptide that is transported by the hpept1 is extracted from the cell layer with 200 ul of a one to one mixture of methanol and water and is counted in 4 ml of CytoScint ES™ scintillation cocktail (ICN). The data are plotted as % Gly-Sar uptake of control (no competitive substrate) versus the competitive substrate concentration. The IC50, defined as that concentration which inhibits 50% of the uptake of the Gly-Sar uptake, indicates the degree of affinity that the test prodrug has for the hpept1. Typically, values that are below 10 mM indicate that the drug interacts with transporter. The results from this experiment using a variety of prodrug compounds is given in Table 1.

TABLE 1
Affinity of prodrugs of acyclovir, ganciclovir, floxuridine, gemcitabine
and melphalan for HPEPT1 in cells that overexpress
the HPEPT1 intestinal transporter

| | DC5 |
|---|---|
| Compound | IC₅₀ mM |
| Val-acyclovir | 0.423 |
| Acyclovir | >25 |
| Val-ganciclovir | 5.23 |
| Ganciclovir | >20 |
| 3′,5′-di-O-phenylalanyl-Floxuridine | 2.78 |
| 3′-O-phenylalanyl-Floxuridine | 3.48 |
| 5′-O-phenylalanyl-Floxuridine | 3.7 |
| 3′,5′-di-O-valyl-Floxuridine | 1.6 |
| 3′-O-valyl-Floxuridine | 0.98 |
| 5′-O-valyl-Floxuridine | 1.17 |
| 3′,5′-di O-prolyl-Floxuridine | >>20 |
| 3′-O-prolyl-Floxuridine | >>20 |
| 5′-O-prolyl-Floxuridine | >>20 |
| 3′,5′-di O-aspartyl-Floxuridine | 9.55 |
| 3′-O-aspartyl-Floxuridine | 10.5 |
| 5′-O-aspartyl-Floxuridine | 8.30 |
| Floxuridine | >>20 |
| 3′,5′ Val-O-Gemcitabine | 1.72 |
| 3′ Val-O-Gemcitabine | 3.69 |
| 5′ Val-O-Gemcitabine | 0.70 |
| 3′,5′ Ile-O-Gemcitabine | 0.82 |
| 3′ Ile-O-Gemcitabine | 2.18 |
| 5′ Ile-O-Gemcitabine | 0.61 |
| Gemcitabine | >>20 |
| Mel-Pro | 0.17 |

The data show that addition of HPEPT1 targeting promoieties to a variety of drugs can improve the affinity of the drug for the HPEPT1 intestinal transporter.

### Example 6

Determination of Prodrug Uptake Mediated by Intestinal Transporter

Hela cells that overexpress hpept1 are incubated with a series of floxuridine prodrugs at a concentration of 50 micromolar in pH 6.0 uptake buffer for 45 minutes. The uptake reaction is stopped by washing of the cells with ice cold PBS three times. The cell layers are collected, the cells lysed, and the amount of parent and prodrug in the cell lysate are determined by high performance liquid chromatography (HPLC). The uptake experiments are repeated in control cultures that do not overexpress the hpept1. The ratio of the test versus control values provides a measure of uptake efficiency for the prodrug by the hpept1 transporter. As seen in Table 2, the 5′ floxuridine and gemcitabine prodrugs show the greatest enhancement of hpept1-mediated uptake. For the floxuridine prodrugs, the phenyl and valyl diester prodrugs show moderate uptake enhancement and the 3′ monoester prodrugs show poor uptake enhancement. For the gemcitabine prodrugs, the 5′ esters of valine and isoleucine showed the greatest enhancement of uptake. For these drugs, the 3′ and 3′,5′ diesters prodrugs showed little or no enhancement of uptake. Stereochemistry was also very important with regard to uptake. Thus, d-amino acids showed virtually no enhancement of uptake.

TABLE 2
Uptake of floxuridine and Gemcitabine Prodrugs in HeLa cells that overexpress HPEPT1.

| | Uptake | | |
|---|---|---|---|
| | (hPepT1) | Uptake Control | |
| A. Anticancer Prodrugs | nmole/mg/45 min | nmole/mg/45 min | hPepT1/Control |
| 3′,5′-di-O-phenylalanyl- | 2.96 ± 0.13* | 0.31 ± 0.02 | 9.53 |
| Floxuridine | | | |
| 3′-O-phenylalanyl-Floxuridine | 0.83 ± 0.19* | 0.32 ± 0.01 | 2.56 |
| 5′-O-phenylalanyl-Floxuridine | 1.58 ± 0.20* | 0.13 ± 0.01 | 12.34 |
| 3′,5′-di-O-valyl-Floxuridine | 2.78 ± 0.37* | 0.54 ± 0.01 | 5.18 |
| 3′-O-valyl-Floxuridine | 1.35 ± 0.13* | 1.32 ± 0.04 | 1.03 |
| 5′-O-valyl-Floxuridine | 3.42 ± 0.09* | 0.18 ± 0.01 | 19.24 |
| Floxuridine | Not detected | Not detected | — |
| 3′-O-L-valyl Gemcitabine | 1.12 ± 0.07 | 1.01 ± 0.03 | 1.11 |
| 5′-O-L-valyl Gemcitabine | 2.14 ± 0.05 | 0.18 ± 0.01 | 11.25 |
| 3′,5′-di-O-L-valyl Gemcitabine | 1.76 ± 0.09 | 1.52 ± 0.05 | 1.15 |
| 3′-O-D-valyl Gemcitabine | 0.81 ± 0.02 | 0.76 ± 0.03 | 1.06 |
| 5′-O-D-valyl Gemcitabine | 1.14 ± 0.04 | 0.72 ± 0.04 | 1.58 |
| 3′,5′-di-O-D-valyl Gemcitabine | 1.11 ± 0.08 | 0.98 ± 0.06 | 1.13 |
| 3′-O-L-isolecucyl Gemcitabine | 1.03 ± 0.11 | 0.94 ± 0.06 | 1.09 |
| 5′-O-L-isolecucyl Gemcitabine | 1.22 ± 0.05 | 0.21 ± 0.01 | 5.64 |
| 3′,5′-di-O-L-isolecucyl | 1.16 ± 0.13 | 1.09 ± 0.03 | 1.06 |
| Gemcitabine | | | |
| 3′-O-L-phenylalanyl | Not detected | Not detected | — |
| Gemcitabine | | | |
| 5′-O-L-phenylalanyl | Not detected | Not detected | — |
| Gemcitabine | | | |
| 3′,5′-di-O-L-phenylalanyl | Not detected | Not detected | — |
| Gemcitabine | | | |
| 3′-O-D-phenylalanyl | Not detected | Not detected | — |
| Gemcitabine | | | |
| 5′-O-D-phenylalanyl | Not detected | Not detected | — |
| Gemcitabine | | | |
| 3′,5′-di-O-D-phenylalanyl | Not detected | Not detected | — |
| Gemcitabine | | | |
| Gemcitabine | Not detected | Not detected | — |
| Valacyclovir | 2.51 ± 0.28 | 0.59 ± 0.06 | 4.25 |

### Example 7

Testing of Floxuridine Prodrugs for Activation with a Prototype Activation Enzyme—BPHL

To look at activation of the amino acid prodrug (e.g., the removal of the amino acid ester), the prodrugs are tested for hydrolysis using the prototype activation enzyme—purified biphenyl hydrolase-like enzyme (BPHL) (Kim I, Chu X Y, Kim S, Provoda C J, Lee K D, Amidon G L—Identification of a human valacyclovirase: biphenyl hydrolase-like protein as valacyclovir hydrolase. J. Biol. Chem. 2003 July 11; 278(28): 25348-56). A solution containing 1 mM of each compound is incubated with the enzyme at 25° C. The reaction is stopped by the addition of 5% trifluoroacetic acid and the amount of parent compound is determined by HPLC analysis. Valacyclovir (VACV) hydrolysis by BPHL is used as a control. As seen in Table 3, the BPHL enzyme showed a range of hydrolytic activity that was dependent upon the linkage (5′ is favored over 3′) and on the identity of the promoiety (valyl>phenylalanine>lysine>aspartic acid).

TABLE 3
Activation of Floxuridine Prodrugs by purified BPHL.

| | % of Control |
|---|---|
| Floxuridine Prodrug | (VACV) |
| 3′,5′ valyl diester Floxuridine | 1.7% |
| 3′ valyl monoester Floxuridine | 3.3% |
| 5′ valyl monoester Floxuridine | 91.1% |
| 3′,5′ phenylalanyl diester Floxuridine | 8.9% |
| 3′ phenylalanyl monoester Floxuridine | 24.4% |
| 5′ phenylalanyl monoester Floxuridine | 52.8% |
| 3′,5′ aspartyl diester Floxuridine | 0.0% |
| 3′ aspartyl monoester Floxuridine | 2.0% |
| 5′ aspartyl monoester Floxuridine | 2.3% |
| 3′,5′ lysyl diester Floxuridine | 0.0% |
| 3′ lysyl monoester Floxuridine | 7.3% |
| 5′ lysyl monoester Floxuridine | 8.1% |
| Valacyclovir (VACV) | 100.0% |

### Example 8

Testing for Activation of Gemcitabine Prodrugs with Intestinal Cell Lysates and Plasma.

Confluent Caco-2 cells are washed with phosphate buffer saline (PBS, pH 7.4) and are harvested with 0.05% Trypsin-EDTA at 37° C. for 5-10 min. Trypsin was neutralized by adding DMEM. The cells are washed off the plate and spun down by centrifugation. The pelleted cells are washed twice with pH 7.4 phosphate buffer (10 mM), and resuspended in pH 7.4 phosphate buffer (10 mM) to obtain a final concentration of approximately 4.70×10⁶ cells/mL. The cells are lysed with one volume 0.5% Triton-X 100 solution. The cell lysate is homogenized by vigorous pipeting and total protein is quantified with the BioRad DC Protein Assay using bovine serum albumin as a standard. The hydrolysis reactions are carried out in 96-well plates (Corning, Corning, N.Y.). Caco-2 cell suspension (230 μl) is placed in triplicate wells and the reactions are started with the addition of substrate and incubated at 37° C. At various time points, 40 μL aliquots are removed and added to two volumes of 10% ice-cold TFA. The mixtures are centrifuged for 10 min at 1800 rcf and 4° C. and the supernatant filtered through a 0.45 μm filter. The recovered filtrate is analyzed by HPLC.

To test stability in human plasma, 230 μL undiluted plasma is added to each well in triplicate and 40 μL of substrate is added to start the reactions which are conducted at 37° C. for up to 4 hours. At various predetermined time points, 40 μL aliquots are removed and added to two volumes of 10% ice-cold TFA. The mixtures are centrifuged for 10 min at 1800 rcf at 4° C. and the supernatant is filtered through a 0.45 μm filter. The recovered filtrate is analyzed by HPLC.

The estimated half-lives (t_{1/2}), obtained from linear regression of pseudo-first-order plots of prodrug concentration vs time are listed in Table 4. The corresponding values for the two reference prodrugs, valacyclovir and valganciclovir are also listed in Table 4. The hydrolysis rates of the gemcitabine prodrugs and the reference prodrugs in plasma were significantly higher in plasma compared to that in phosphate buffer, pH 7.4. the hydrolysis rates of the prodrugs in Caco-2 cell homogenates are roughly comparable to that seen in plasma. These enhanced rates of degradation suggest specific enzymatic action. Two effects are noted: a) the effect of structure of promoiety on stability was in the order, isoleucyl>valyl>>phenylalanyl; and b) the stereochemistry of the promoiety affected the stability of the gemcitabine prodrugs in a profound manner (D-valyl and D-phenylalanyl prodrugs were roughly 4- to 14-fold more stable in Caco-2 cell homogenates than the corresponding L-analog).

TABLE 4
Activation of Gemcitabine prodrugs in buffer, caco-2 cell
homogenates and Human plasma.

| | t_{1/2} (min) | | |
|---|---|---|---|
| | | Human | Caco-2 cell |
| Prodrug | Buffer pH 7.4 | plasma | homogenates |
| 3′-O-L-valyl gemcitabine | 64.0 ± 1.4 | 5.4 ± 0.1 | 5.0 ± 0.1 |
| 5′-O-L-valyl gemcitabine | 416.0 ± 8.5 | 56.4 ± 2.9 | 7.1 ± 0.6 |
| 3′,5′-di-O-L-valyl | 55.0 ± 2.7 | 2.0 ± 0.1 | 0.9 ± 0.0 |
| gemcitabine | | | |
| 3′-O-D-valyl gemcitabine | 74.0 ± 1.2 | 5.99 ± 0.0 | 23.2 ± 0.2 |
| 5′-O-D-valyl gemcitabine | 424.0 ± 1.2 | 58.1 ± 2.1 | 37.4 ± 1.4 |
| 3′,5′-di-O-D-valyl | 52.0 ± 1.1 | 2.1 ± 0.1 | 10.3 ± 0.7 |
| gemcitabine | | | |
| 3′-O-L-isoleucyl | 66.0 ± 0.21 | 8.0 ± 0.1 | 10.6 ± 0.3 |
| gemcitabine | | | |
| 5′-O-L-isoleucyl | 452.0 ± 9.6 | 99.2 ± 1.6 | 75.3 ± 2.8 |
| gemcitabine | | | |
| 3′,5′-di-O-L-isoleucyl | 61.0 ± 0.5 | 2.64 ± 0.0 | 2.1 ± 0.0 |
| gemcitabine | | | |
| 3′-O-L-phenylalanyl | 39.0 ± 0.12 | 5.7 ± 0.1 | 0.8 ± 0.0 |
| gemcitabine | | | |
| 5′-O-L-phenylalanyl | 200.0 ± 1.9 | 8.4 ± 0.2 | 3.2 ± 0.1 |
| gemcitabine | | | |
| 3′,5′-di-O-L-phenylalanyl | 38.0 ± 0.2 | 0.7 ± 0.0 | 0.6 ± 0.0 |
| gemcitabine | | | |
| 3′-O-D-phenylalanyl | 39.0 ± 0.7 | 7.7 ± 0.2 | 8.8 ± 0.1 |
| gemcitabine | | | |
| 5′-O-D-phenylalanyl | 204.0 ± 3.5 | 34.8 ± 1.1 | 11.4 ± 0.2 |
| gemcitabine | | | |
| 3′,5′-di-O-D-phenylalanyl | 28.0 ± 0.6 | 2.4 ± 0.2 | 8.3 ± 0.6 |
| gemcitabine | | | |
| Valacyclovir | 1029.0 ± 11.4 | 312.0 ± 24.6 | 27.7 ± 0.6 |
| Valganciclovir | 990.0 ± 14.4 | 303.0 ± 18.0 | 32.7 ± 0.7 |

Any patents or publications mentioned in this specification are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The apparatus and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A prodrug composition comprising:
a pharmaceutical species characterized by bioavailability of 30% or less, wherein the pharmaceutical species has a molecular weight in the range of 100-1000 Daltons, and with the proviso that the pharmaceutical species is not acyclovir, ganciclovir, BRL44385, or penciclovir; and
an amino acid having a covalent bond to the pharmaceutical species.

2. The composition of claim 1 wherein the amino acid is selected from the group consisting of: an α-amino acid, a β-amino acid and a γ-amino acid.

3. The composition of claim 2 wherein the amino acid is a naturally occurring amino acid

4. The composition of claim 2 wherein the amino acid is an L-amino acid.

5. The composition of claim 2 wherein the amino acid is a non-polar amino acid.

6. The composition of claim 3 wherein the amino acid is valine.

7. The composition of claim 1 further comprising a second amino acid covalently coupled to the pharmaceutical species.

8. The composition of claim 1 wherein the pharmaceutical species has a molecular weight in the range of 260-800 Daltons.

9. The composition of claim 1 wherein the pharmaceutical species is selected from the group consisting of: floxuridine, gemcitabine, cladribine, melphalan, cis-platin, cytarabine, fludarabine, cidofovir, tenofovir, pentostatin, dacarbazine, mercaptopurine, thioguanine, adenocard, adriamycin, allopurinol, alprostadil, amifostine, aminohippurate, argatroban, benztropine, bortezomib, busulfan, calcitriol, carboplatin, daunorubicin, dexamethasone, topotecan, docetaxel, dolasetron, doxorubicin, epirubicin, estradiol, famotidine, foscarnet, flumazenil, fosphenytoin, fulvestrant, hemin, ibutilide fumarate, irinotecan, levocarnitine, idamycin, sumatriptan, granisetron, metaraminol, metaraminol, methohexital, mitoxantrone, morphine, nalbuphine hydrochloride, Nesacaine, oxaliplatin, palonosetron, pamidronate, pemetrexed, phytonadione, ranitidine, testosterone, tirofiban, toradol, triostat, valproate, vinorelbine tartrate, visudyne, zemplar, zemuron, and zinecard.

10. The composition of claim 1 wherein the covalent bond creates a moiety between the pharmaceutical species and the amino acid that is an amide.

11. The composition of claim 1 wherein the covalent bond creates a moiety between the pharmaceutical species and the amino acid that is an ester.

12. The composition of claim 1 wherein the covalent bond creates a moiety between the pharmaceutical species and the amino acid that is an oxime.

13. The composition of claim 1 wherein the covalent bond creates a moiety between the pharmaceutical species and the amino acid that is selected from the group consisting of: an ether, a secondary amine, and a tertiary amine.

14. The composition of claim 1 wherein the prodrug is formulated as a pharmaceutically acceptable salt.

15. The composition of claim 1 wherein pharmaceutical species comprises a halogen.

16. The composition of claim 1 characterized by enhanced bioavailability of the pharmaceutical species when the composition is orally administered to an individual.

17. The composition of claim 16 wherein the enhanced bioavailability is two fold or more.

18. A prodrug composition comprising:
a pharmaceutical species selected from the group consisting of: floxuridine, gemcitabine, cladribine, melphalan, and cidofovir; and
an amino acid having a covalent bond to the pharmaceutical species.

19. The composition of claim 18 wherein the amino acid is selected from the group consisting of: an α-amino acid, a β-amino acid and a γ-amino acid.

20. The composition of claim 19 wherein the amino acid is a naturally occurring amino acid.

21. The composition of claim 20 wherein the amino acid is an L-amino acid.

22. The composition of claim 19 wherein the amino acid is a non-polar amino acid.

23. The composition of claim 20 wherein the amino acid is valine.

24. The composition of claim 18 wherein the covalent bond creates a moiety between the pharmaceutical species and the amino acid that is selected from the group consisting of: an amide, an ester, and an oxime.

25. The composition of claim 18 wherein the covalent bond creates a moiety between the pharmaceutical species and the amino acid that is selected from the group consisting of: an ether, a secondary amine, and a tertiary amine.

26. The composition of claim 18 wherein the prodrug is formulated as a pharmaceutically acceptable salt.

27. The composition of claim 18 characterized by enhanced bioavailability of the pharmaceutical species when the composition is orally administered to an individual.

28. The composition of claim 18 further comprising a second amino acid covalently coupled to the pharmaceutical species.

29. A method of delivering a pharmaceutical species to an individual, comprising the step of:
orally administering a prodrug to the gastrointestinal lumen of an individual, the prodrug comprising a pharmaceutical species characterized by bioavailability of 30% or less, wherein the pharmaceutical species has a molecular weight in the range of 100-1000 Daltons, and with the proviso that the pharmaceutical species is not acyclovir, ganciclovir, BRL44385, or penciclovir; and an amino acid having a covalent bond to the pharmaceutical species, wherein the prodrug is transported from the gastrointestinal lumen by a specific transporter and is enzymatically cleaved to yield the pharmaceutical species, thereby delivering the pharmaceutical species to the individual.

30. The method of claim 29 wherein the pharmaceutical species is selected from the group consisting of: floxuridine, gemcitabine, cladribine, melphalan, cis-platin, cytarabine, fludarabine, cidofovir, tenofovir, pentostatin, dacarbazine, mercaptopurine, thioguanine, adenocard, adriamycin, allopurinol, alprostadil, amifostine, aminohippurate, argatroban, benztropine, bortezomib, busulfan, calcitriol, carboplatin, daunorubicin, dexamethasone, topotecan, docetaxel, dolasetron, doxorubicin, epirubicin, estradiol, famotidine, foscarnet, flumazenil, fosphenytoin, fulvestrant, hemin, ibutilide fumarate, irinotecan, levocarnitine, idamycin, sumatriptan, granisetron, metaraminol, metaraminol, methohexital, mitoxantrone, morphine, nalbuphine hydrochloride, nesacaine, oxaliplatin, palonosetron, pamidronate, pemetrexed, phytonadione, ranitidine, testosterone, tirofiban, toradol, triostat, valproate, vinorelbine tartrate, visudyne, zemplar, zemuron, and zinecard.

31. The method of claim 29 wherein the prodrug is formulated as a pharmaceutically acceptable salt.
